(19) United States
(12) Patent Application Publication
(10) Pub. No.: US 2008/0187907 A1
(43) Pub. Date: Aug. 7, 2008

(54) Catalytic Production of Biomakers From Biological Materials

(76) Inventors: Calvin H. Bartholomew, Orem, UT (US); Zhijun Jia, Provo, UT (US); Phillip R. Smith, Provo, UT (US); Aaron Nicholas Nackos, Provo, UT (US); Milton L. Lee, Pleasant Grove, UT (US); Edgar D. Lee, Highland, UT (US)

Correspondence Address: JAMES SONNTAG;JAMES SONNTAG, PATENT ATTORNEY, P.O. BOX 9194, SALT LAKE CITY, UT, 84109, US

(21) Appl. No.: 10/590,545

(22) PCT Filed: Feb. 25, 2005
(86) PCT No.: PCT/US05/06250

§ 371 Date: Sep. 27, 2007

Related U.S. Application Data
(60) Provisional application No. 60/547,950, filed on Feb. 26, 2004.

(51) Int. Cl. C12Q 1/68; C12Q 1/04; C12Q 1/00

(52) U.S. Cl. 435/6; 435/34; 435/4

(57) ABSTRACT

An apparatus and method for detecting a biological material, such as bacterial spores (e.g. Anthrax) which reacts non-volatile biomarker precursors to volatile precursors by heating the biological material in the presence of a catalyst.

### CROSS REFERENCE TO RELATED APPLICATIONS

This application claims priority from U.S. Provisional Patent Application 60/547950, filed 26 Feb. 2004.

### FEDERAL RESEARCH STATEMENT

This invention was made with support from United States Government, and the United States Government may have certain right in this invention pursuant to Defense Threat Reduction Agency Contract Number DTRA 01-03-C-0047.

### BACKGROUND OF INVENTION

Handheld methods and instrumentation for rapid identification of unknown biological samples are needed in many different areas, including medical diagnostics, forensic investigations, microbiological research, civil defense, and military operations. This technology is not currently available. One of the most important applications for civil and military defense is the detection and identification of biological warfare agents. This application is essential to the national security of the United States of America.

Of particular concern is the weaponized form of the bacterial agent Bacillus anthracis, commonly known as anthrax [1-3]. Anthrax can be lethal in very small doses (8,000-10,000 spores, or about 10 nanograms), making it an extremely potent biological weapon [3, 4]. The toxicity of anthrax, combined with the ease of dispersal and large atmospheric residence times, makes it a very dangerous biological weapon. This was illustrated recently by the deaths of several civilians due to contact with anthrax endospores sent through the US Postal Service [5, 6]. The discovery of a suspicious white powder was followed by several days of tests, and finally the conclusion that the material was anthrax [6]. More rapid methods for the detection and identification of anthrax are therefore crucial in order to prevent/defend against anthrax attacks and facilitate a rapid response to mitigate its effects [7]. The US military is especially interested in technologies that can rapidly detect the presence of anthrax, to (a) protect its armed forces from biological attack and (b) track down and stop terrorists and/or rogue states that are producing or developing biological warfare agents [3]. The technology required for the detection and identification of anthrax spores is representative of that required for the detection of many other types of biological materials.

### Detection and Identification of Biological Warfare Agents

Historically, the method of choice for identifying an unknown sample of bacterial origin was to grow colonies from the spores; following the culture growth, solution assays, stains, and microscopic visualization were used to confirm the presence of anthrax in the original sample [8]. While this approach works, it takes days to accomplish, and requires significant amounts of equipment and personnel. Therefore, there have been significant efforts over the last 40 years to develop novel, more rapid methods.

Much effort has been focused on ways to use the many different biochemical compounds contained in bacterial spores in identification algorithms. Methods used to extract these biochemical compounds from the microorganism and convert them to detectible chemicals (biomarkers) play a key role in the detection technology. Typical biomarker precursors include fatty acids, proteins, carbohydrates, and/or deoxyribonucleic acid (DNA); for some organisms specific chemicals such as calcium complexed-dipicolinic acid (DPA) may be important (e.g. in bacterial spores DPA accounts for 5-15% of the dry weight).

Several methods and devices to rapidly and reproducibly generate biomarkers from bacterial spores have been developed over the past three decades, although it must be stated that generally they remain in the developmental stage. Commercially available detection systems are expensive and exhibit limited utility. They include the separate technologies utilized with point detection (i.e., detection on-site), standoff technologies (on-site sample retrieval and subsequent analysis off-site), and passive standoff detection (complete detection is performed without any physical interaction with a sample, such as in spectroscopic methods) [8a]. Wet and dry point detection methods are used. Wet methods are usually biological interaction-based (e.g. antibody recognition), while dry detection methods are utilized to physically decompose a sample and detect the chemical fragments that are released. Both wet and dry methods require sample preparation, followed by detection. For example, the sequencing of DNA gleaned from cellular extracts can be used for the unique identification of bacteria, including anthrax [9]. Unfortunately, this method takes hours and requires very specialized equipment that is not easily miniaturized, and cannot be applied to spores. Analytical pyrolysis, however, which breaks down and/or converts the biomarker precursors to more volatile biomarkers, has become a viable approach for rapid identification of biological materials, although there are limits to its portability.

### Analytical Pyrolysis

Pyrolysis is defined as the breaking of chemical bonds by thermal energy. It has found application in the analysis of polymers and other high molecular weight compounds [10-12]. During pyrolysis, there are two major classifications of chemical reactions that occur, primary and secondary. The primary reactions typically involve the thermal decomposition of low-and high-molecular weight compounds. Ideally, these compounds are swept into the detector rapidly, without reacting further. In practice, secondary reactions may occur; for example, primary products may react with the walls of the reactor or other molecules such as oxygen or other primary pyrolysis products [10-13]. To avoid these problems, decomposition reactions and analytical devices must be closely coupled. Analytical pyrolysis (AP) is the close coupling of pyrolysis with analytical chemistry techniques, allowing the detection and identification of the compounds produced during pyrolysis. Useful, semi-portable analytical techniques are typically gas chromatography (GC) and mass spectrometry (MS).

In most AP methods, biological polymers (protein, peptidoglycan, and DNA) are broken down and/or converted to more volatile compounds. In their naturally occurring state, the biomarker precursors are of sufficiently low volatility to preclude detection by standard analytical techniques; however, if chemically changed to a more volatile state, these compounds, or biomarkers, can be more easily detected. This is realized by rapidly heating the sample to elevated temperatures, i.e. 350-650° C. [13, 14]. The first use of pyrolytically-produced biomarkers for the detection of bacteria was reported over 30 years ago; it has been a subject of continuous research since then and is still a subject of intense research today [15-18]. A major product observed during the pyrolysis of gram-positive bacterial spores is picolinic acid, one of the primary pyrolysis products of dipicolinic acid [19, 20]. Other compounds observed during AP include decomposition products of protein and peptidoglycan, including diketopiperazines or other cyclized oligopeptides, from which many of the amino acid side groups have been cleaved [21]. Oligopeptide cyclization is an example of a secondary pyrolytic reaction [22, 23].

There are two general classes of AP that have been used to produce biomarkers from bacterial spores. First, curie-point pyrolysis utilizes an inductively heated fine wire to pyrolyze a dried biological sample. This method has been successful for differentiating bacteria at the gram-classification level. Second, thermal-hydrolysis methylation (THM) utilizes a methylating agent in the pyrolyzer to derivatize the fatty acids. Tetramethylammonium hydroxide has been widely accepted as the methylating agent of choice. This method has been able to differentiate bacteria at the species and even strain level. In both of these methods, analysis times have improved significantly, some to less than 15 minutes. Each of these methods is discussed in turn.

Curie-Point Pyrolysis

Snyder et al. have developed a pyrolytic method to remove and volatilize DPA from the inside of the endospore [13, 17, 19, 20, 24, 25]. Following collection of aerosolized spores on a quartz frit filter or deposition of liquid spore suspensions on a small Curie-point wire, they used high temperature pyrolysis (350-600° C.) to free DPA from the spores. The DPA in the pyrolyzate was analyzed by gas-chromatography-mass spectrometry (GC-MS), with the analysis and detection times measured in minutes. Although this analysis is reasonably fast, it requires high temperatures, a large energy expenditure, and very specialized equipment. Moreover, the pyrolysis generates numerous by-products, i.e. there were many side reactions as exemplified in FIG. 1, which shows a reaction pathway for pyrolytic degradation and electron impact fragmentation pathway of dipicolinic acid [19]. The by-products complicate the pyrograms and data analysis. Sophisticated pattern recognition algorithms are employed to aid in interpretation of the data, which increases the complexity of the system and requires additional computer hardware and software (i.e. renders the system less portable).

Recently, Snyder et al. have assessed the microbiological meaning (chemotaxonomy) of the specific biomarkers produced by curie-point pyrolysis [17]. The biomarkers were detected by GC-IMS (Ion Mobility Spectrometry) and identified by comparison with both the National Institute of Standards and Technology (NIST) database and analytical standards. The list of compounds presented in Table I shows the biomarkers detected and identified by Snyder et al. They concluded that some biomarkers might be produced but are not observed due to inefficient heating and flow paths in their device. The curie-point pyrolysis method developed by Snyder et al. has demonstrated capability for differentiating bacteria and bacterial spores at the gram-classification level, but has been unsuccessful at the differentiation of anthrax from closely related species.

TABLE I
Biomarkers Produced and Detected by Snyder et al. (2004) [17].

| Peak | | | |
|---|---|---|---|
| Number | Structure | Name | Source |
| 1 N, S | 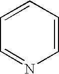 | Pyridine | Picolinic acid,Proteins,Nucleic acids |
| 2 N, S | 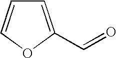 | 2-furan-carboxaldehyde, [symbol] | Carbohydrates |
| 3 N, S | 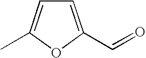 | 5-methyl-2- [symbol] aldehyde, 5-methyl- [symbol] | Carbohydrates |
| 4 N, S | 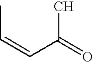 | Crobonic acid | Poly (3-hydroxy- [symbol] acid),Gram + |
| 5 N, S | 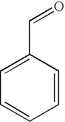 | Benzene- [symbol] aldehyde,benzaldehyde | Proteins |
| 6 N | 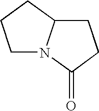 | [symbol] one | Proteins |
| 7 N, S | 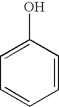 | Phenol | Proteins: lyrosine |
| 8 N, S | 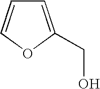 | 2-hydroxymethylfuran, [symbol] alcohol | Nucleic acids,carbohydrates |
| 9 N, S | 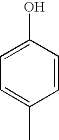 | p-Cresol | Proteins |
| 10 N, S | 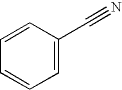 | Phenylacetonitrile,benzonitrile | Proteins |
| 11 N, S | 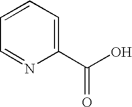 | Pyridine-2-carboxylic acid,picolinic acid | Pyridine-2,5-dicarboxylic acid,Gram positive sporos |
| 12 N | 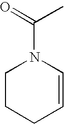 | 1-acetyl-1,2,3,4-tetrahydropyridine | Proteins |
| 13 N | 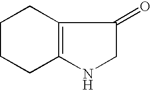 | 4,5,6,7-tetrahydroindole-3-one | Proteins |
| 14 N | 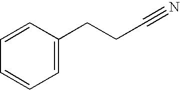 | Benzenepropanenitrile | Proteins |
| 15 N, S | 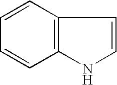 | Indole | Proteins |
| 16 | (CH₂)₃CH(CH₃)₂CH | iso-tridecanenitrile | Branched lipids;lipoproteins |
| 17 | CH₂(CH₂)₁₀CH—CH₃ | 1-tridecane | Lipopolysaccharides(LPE), Gram − |
| 18 N, S | 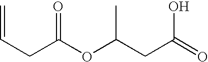 | Diener of crolonicacid | Poly (3-hydroxy-butyric acid),Gram + |
| 19 N, S | 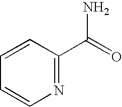 | 2-pyridine-carboxamide,picolinamide | Peptidoglycan,Gram + |
| 20 N, S | 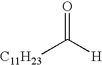 | Dodecanal | LPS, Gram − |
| 21 | 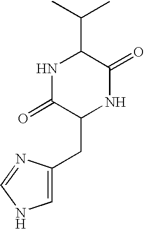 | Dikelopiperazine(His-Val) | Proteins |
| 22 N, S | 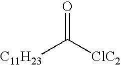 | 2-tridecanone | LPS, Gram − |
| 23 N, S | 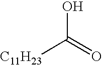 | n-dodocanoic acid,lauric acid | Phospholipids,LPS, Gram − |
| 24 | 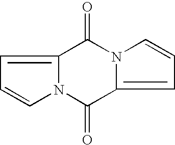 | Dikelopiperazine(Pro-Pro) | Proteins |
| 25 N | 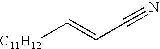 | 2-tetradocenitrile | LPS, Gram − |
| 26 N, S | 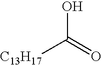 | n-tetradocenoicacid, myristic acid | Phospholipids,LPS, Gram − |

[symbol]

indicates data missing or illegible when filed

Thermal Hydrolysis-Methylation

It has long been known that the lipid contents of bacterial spores contain a significant amount of taxonomical information that could be exploited to differentiate anthrax spores from those of closely related species [26, 27]. However, lipids are very sticky, non-volatile compounds, and are by themselves not readily analyzed by GC and/or MS. Traditionally, chemical extraction methods have been used to remove the free lipids from the spores; the lipids are then derivatized (methylated) in vitro to create fatty acid methyl esters (FAMEs) that are volatile enough to be detected by GC or MS [15, 28]. This application has been commercialized by MIDI, Inc, 125 Sandy Drive, Newark, Del. 19713 (www.midi-inc.com) and features an automated system for the chemical extraction and derivatization of the fatty acids; a pyrolysis unit is used to volatilize the FAMEs for analysis by GC-MS.

More recently, a process called thermal hydrolysis-methylation (THM) has been developed which is capable of not only methylating the free fatty acids with a powerful methylation agent, for example, tetramethylammonium hydroxide, (TMAH), but also transesterifying the bound fatty acids; this increases the amount of information available in the lipid profile [29-44]. THM is typically conducted in situ at high temperatures in a pyrolyzer similar to that used for analytical pyrolysis. THM, it should be emphasized, is a non catalytic method, since the methylation agent TMAH is consumed in the process, and this process is driven by thermal decomposition and rearrangement of chemical bonds.

Voorhees et al. have developed methods and devices to produce fatty acid methyl esters (FAMEs) from spore lipids by THM [29-39, 42, 43]. The FAMEs are typically analyzed by GC/MS or direct MS to construct a lipid profile. Pattern recognition algorithms have been employed to interpret these profiles to confirm the presence or absence of anthrax [38, 39, 41, 45]. They have shown that FAME profiles are unique for each bacterial species and thereby facilitate potentially unambiguous identification [38]. Recently Havey et al. at Sandia National Laboratories have collaborated with Voorhees et al. to develop a ceramic membrane heating system that is capable of heating to over 200° C. in milliseconds to generate FAMEs from bacterial spores. This device has very low power (milli-watt) requirements, but has yet to be field-tested or thoroughly evaluated [43].

Other derivatization methods very similar to thermal hydrolysis methylation have been proposed as a means to profile the carbohydrates in bacteria [46]. However, field portable devices have not been developed for this approach.

### Limitations of Analytical Pyrolysis

While these recent developments in analytical pyrolysis have resulted in faster biomarker production and detection times, the required equipment tends to be bulky and to require relatively large amounts of power. Also, reproducibility and general applicability of biomarker generation techniques are lacking and have not been well addressed in the literature. In order to develop handheld devices for the rapid detection of anthrax, further advancements of this technology are needed. Improvements in biomarker production speed and reproducibility as well as reductions in detection time, analytical sophistication, equipment size, and power consumption are all necessary to advance the technology to a field-portable, handheld level.

### CITED REFERENCES

1. Inglesby, T. V., et al., Anthrax as a Biological Weapon, 2002—Updated Recommendations for Management. Jama-Journal of the American Medical Association, 2002. 287(17): p. 2236-2252.

2. Inglesby, T. V., Anthrax as a Biological Weapon, 2002: Updated Recommendations for Management (vol 287, pg 2236, 2002). Jama-Journal of the American Medical Association, 2002. 288(15): p. 1849-1849.

3. Hawley, R. J. and E. M. Eitzen Jr, Biological Weapons—A Primer for Microbiologists. Annual Review of Microbiology, 2001. 55(1): p. 235-253.

4. Pepper, I. L. and T. J. Gentry, Incidence of Bacillus anthracis in Soil. Soil Science, 2002. 167(10): p. 627-635.

5. Weis, C. P., et al., Secondary Aerosolization of Viable Bacillus anthracis Spores in a Contaminated US Senate Office. Jama-Journal of the American Medical Association, 2002. 288(22): p. 2853-2858.

6. Parker, J. S., Terrorism Through the Mail: Protecting the Postal Workers and the Public, in Report Submitted to the Committee on Governmental Affairs and the Subcommittee on International Security, Proliferation, and Federal Service. 2001, U.S. Army Medical Research and Materiel Command and Fort Detrick.

7. Birenzwige, A. and C. Wick, US Army Report ECBC-TR-255. US Army Report, 2002.

8. Jackson, P. J., et al., PCR Analysis of Tissue Samples from the 1979 Sverdlovsk Anthrax Victims: The Presence of Multiple Bacillus anthracis Strains in Different Victims. Proceedings of the National Academy of Sciences of the United States of America, 1998. 95(3): p. 1224-1229.

8a . National Institute of Justice, An Introduction to biological agent detection equipment for emergency first responders. U.S. Department of Justice, December, 2001.

9. Bell, C. A., et al., Detection of Bacillus anthracis DNA by LightCycler PCR. Journal of Clinical Microbiology, 2002. 40(8): p. 2897-2902.

10. Wampler, T. P., Applied Pyrolysis Handbook. 1995, New York: M. Dekker. 361 p.

11. Wampler, T. P., Introduction to Pyrolysis-Capillary Gas Chromatography. Journal of Chromatography A, 1999. 842(1-2): p. 207-220.

12. Wampler, T. P., Practical applications of analytical pyrolysis. Journal of Analytical and Applied Pyrolysis, 2004. 71(1): p. 1-12.

13. Snyder, A. P., et al., Field Detection and Identification of a Bioaerosol Suite by Pyrolysis-Gas Chromatography-Ion Mobility Spectrometry. Field Analytical Chemistry and Technology, 2001. 5(4): p. 190-204.

14. Snyder, A. P., et al., Pyrolysis-Gas Chromatography-Mass Spectrometry—Detection of Biological Warfare Agents, in Mass Spectrometry for the Characterization of Microorganisms. 1994, AMERICAN CHEMICAL SOCIETY: Washington. p. 62-84.

15. Tabor, M. W., J. MacGee, and J. W. Holland, Rapid Determination of Dipicolinic Acid in the Spores of Clostridium Species by Gas-Liquid Chromatography. Applied and Environmental Microbiology, 1976. 31(1): p. 25-8.

16. Irwin, W. J., Analytical Pyrolysis: a Comprehensive Guide. Chromatographic science; v. 22. 1982, New York, N.Y.: M. Dekker. 578 p.

17. Snyder, A. P., et al., Correlation of Mass Spectrometry Identified Bacterial Biomarkers from a Fielded Pyrolysis-Gas Chromatography-Ion Mobility Spectrometry Biodetector with the Microbiological Gram Stain Classification Scheme. Analytical Chemistry, 2004. 76(21): p. 6492-6499.

18. Dworzanski, J. P., et al., Novel biomarkers for Gram-type differentiation of bacteria by pyrolysis-gas chromatography-mass spectrometry. Journal of Analytical and Applied Pyrolysis, 2005. In Press, Corrected Proof.

19. Goodacre, R., et al., Detection of the Dipicolinic Acid Biomarker in Bacillus Spores Using Curie-Point Pyrolysis Mass Spectrometry and Fourier Transform Infrared Spectroscopy. Analytical Chemistry, 2000. 72(1): p. 119-127.

20. Snyder, A. P., et al., Detection of the Picolinic Acid Biomarker in Bacillus Spores Using a Potentially Field-Portable Pyrolysis Gas Chromatography Ion Mobility Spectrometry System. Field Analytical Chemistry and Technology, 1996. 1(1): p. 49-59.

21. Voorhees, K. J., et al., An Investigation of the Pyrolysis of Oligopeptides by Curie-Point Pyrolysis Tandem Mass-Spectrometry. Journal of Analytical and Applied Pyrolysis, 1994. 30(1): p. 1-16.

22. Hendricker, A. D., F. Basile, and K. J. Voorhees, A Study of Protein Oxidative Products Using a Pyrolysis-Membrane Inlet Quadrupole Ion Trap Mass Spectrometer with Air as the Buffer Gas. Journal of Analytical and Applied Pyrolysis, 1998. 46(1): p. 65-82.

23. Hendricker, A. D. and K. J. Voorhees, Amino Acid and Oligopeptide Analysis Using Curie-Point Pyrolysis Mass Spectrometry with In-Situ Thermal Hydrolysis and Methylation: Mechanistic Considerations. Journal of Analytical and Applied Pyrolysis, 1998. 48(1): p. 17-33.

24. Snyder, A. P., Quartz Tube, Curie-Point Wire and Ribbon Filament Pyrolyzate Characterization of Biological Materials with Gas Chromatography and Mass Spectrometry Techniques. 1992, Chem. Res., Dev. Eng. Cent., Aberdeen Proving Ground, Md., USA. p. 187 pp.

25. Snyder, A. P., et al., Field Detection of Bacillus Spore Aerosols with Stand-Alone Pyrolysis-Gas Chromatography-Ion Mobility Spectrometry. Field Analytical Chemistry and Technology, 1999. 3(4-5): p. 315-326.

26. Shaw, N., Lipid Composition as a Guide to the Classification of Bacteria. Advances in Applied Microbiology, 1974. 17(0): p. 63-108.

27. Kossa, W. C., et al., Pyrolytic methylation/gas chromatography: A short review. Journal of Chromatographic Science, 1979. 17(4): p. 177-87.

28. MIDI, Sherlock® Microbial Identification Systems. 2004.

29. Kurkiewicz, S., et al., GC/MS Determination of Fatty Acid Picolinyl Esters by Direct Curie-Point Pyrolysis of Whole Bacterial Cells. Journal of the American Society for Mass Spectrometry, 2003. 14(1): p. 58-62.

30. Deluca, S., et al., Direct Analysis of Bacterial Fatty-Acids by Curie-Point Pyrolysis Tandem Mass-Spectrometry. Analytical Chemistry, 1990. 62(14): p. 1465-1472.

31. Deluca, S. J., E. W. Sarver, and K. J. Voorhees, Direct Analysis of Bacterial Glycerides by Curie-Point Pyrolysis Mass-Spectrometry. Journal of Analytical and Applied Pyrolysis, 1992. 23(1): p. 1-14.

32. Basile, F., et al., Pathogenic Bacteria: Their Detection and Differentiation by Rapid Lipid Profiling with Pyrolysis Mass Spectrometry. Trac-Trends in Analytical Chemistry, 1998. 17(2): p. 95-109.

33. Beverly, M. B., et al., A Rapid Approach for the Detection of Dipicolinic Acid in Bacterial Spores Using Pyrolysis Mass Spectrometry. Rapid Communications in Mass Spectrometry, 1996. 10(4): p. 455-458.

34. Beverly, M. B., K. J. Voorhees, and T. L. Hadfield, Direct Mass Spectrometric Analysis of Bacillus Spores. Rapid Communications in Mass Spectrometry, 1999. 13(23): p. 2320-2326.

35. Voorhees, K. J., S. J. Deluca, and A. Noguerola, Identification of Chemical Biomarker Compounds in Bacteria and Other Biomaterials by Pyrolysis Tandem Mass-Spectrometry. Journal of Analytical and Applied Pyrolysis, 1992. 24(1): p. 1-21.

36. Voorhees, K. J., et al., The Use of Biomarker Compounds for the Identification of Bacteria by Pyrolysis Mass Spectrometry. Journal of Analytical and Applied Pyrolysis, 1997. 40-1: p. 111-134.

37. Luo, S., et al., Study of Purified Bacteria and Viruses by Pyrolysis Mass Spectrometry. Field Analytical Chemistry and Technology, 1999. 3(6): p. 357-374.

38. Hendricker, A. D., et al., Rapid Chemotaxonomy of Pathogenic Bacteria Using In Situ Thermal Hydrolysis and Methylation as a Sample Preparation Step Coupled with a Field-Portable Membrane-Inlet Quadrupole Ion Trap Mass Spectrometer. International Journal of Mass Spectrometry, 1999. 191: p. 331-342.

39. Madonna, A. J., K. J. Voorhees, and T. L. Hadfield, Rapid Detection of Taxonomically Important Fatty Acid Methyl Ester and Steroid Biomarkers Using In Situ Thermal Hydrolysis/Methylation Mass Spectrometry (THM-MS): Implications for Bioaerosol Detection. Journal of Analytical and Applied Pyrolysis, 2001. 61(1-2): p. 65-89.

40. Griest, W. H., et al., Biological Agent Detection and Identification by the Block II Chemical Biological Mass Spectrometer. Field Analytical Chemistry and Technology, 2001. 5(4): p. 177-184.

41. Xu, M., F. Basile, and K. J. Voorhees, Differentiation and Classification of User-Specified Bacterial Groups by In Situ Thermal Hydrolysis and Methylation of Whole Bacterial Cells with tert-Butyl Bromide Chemical Ionization Ion Trap Mass Spectrometry. Analytica Chimica Acta, 2000. 418(2): p. 119-128.

42. Xu, M., K. J. Voorhees, and T. L. Hadfield, Repeatability and Pattern Recognition of Bacterial Fatty Acid Profiles Generated by Direct Mass Spectrometric Analysis of In Situ Thermal Hydrolysis/Methylation of Whole Cells. Talanta, 2003. 59(3): p. 577-589.

43. Havey, C. D., et al., Evaluation of a Micro-Fabricated Pyrolyzer for the Detection of Bacillus anthracis Spores. Journal of Analytical and Applied Pyrolysis, 2004. 72(1): p. 55-61.

44. Basile, F., et al., Direct Mass Spectrometric Analysis of In Situ Thermally Hydrolyzed and Methylated Lipids from Whole Bacterial Cells. Analytical Chemistry, 1998. 70(8): p. 1555-1562.

45. Harrington, P. D., et al., Validation Using Sensitivity and Target Transform Factor Analyses of Neural Network Models for Classifying Bacteria from Mass Spectra. Journal of the American Society for Mass Spectrometry, 2002. 13(1): p. 10-21.

46. Gilbart, J., A. Fox, and S. L. Morgan, Carbohydrate Profiling of Bacteria by Gas Chromatography-Mass Spectrometry—Chemical Derivatization and Analytical Pyrolysis. European Journal of Clinical Microbiology & Infectious Diseases, 1987. 6(6): p. 715-723.

47. Farrauto, R. J. and C. Bartholomew, Fundamentals of Industrial Catalytic Processes. 1st ed. 1997, London: Blackie Academic and Professional. 552 pp (approx ).

48. Timofeeva, M. N., et al., Esterification of 2,6-Pyridinedicarboxylic Acid with N-Butanol Catalyzed by Heteropoly Acid H3PW12040 or Its Ce(Iii) Salt. Journal of Molecular Catalysis A-Chemical, 1995. 102(2): p. 73-77.

49. Timofeeva, M. N., et al., Esterification of n-Butanol with Acetic Acid in the Presence of Heteropoly Acids, with Different Structures and Compositions. Kinetics and Catalysis, 2001. 42(6): p. 791-795.

50. Timofeeva, M. N., Acid Catalysis by Heteropoly Acids. Applied Catalysis A-General, 2003. 256(1-2): p. 19-35.

51. Timofeeva, M. N., et al., Esterification of n-Butanol with Acetic Acid in the Presence of H3PW12040 Supported on Mesoporous Carbon Materials. Kinetics and Catalysis, 2003. 44(6): p. 778-787.

52. Bondioli, P., The Preparation of Fafty Acid Esters by Means of Catalytic Reactions. Topics in Catalysis, 2004. 27(1-4): p. 77-82.

53. Loison, R. and R. Chauvin, Chim. Ind., 1964. 91(269).

54. Jamil, K., J. I. Hayashi, and C. Z. Li, Pyrolysis of a Victorian brown coal and gasification of nascent char in CO2 atmosphere in a wire-mesh reactor. Fuel, 2004. 83(7-8): p. 833-843.

55. Gonenc, Z. S., et al., Comparison Of Coal Pyrolysis Product Distributions From 3 Captive Sample Techniques. Fuel, 1990. 69(3): p. 383-390.

56. Gibbins, J. R., et al., Variable-Heating-Rate Wire-Mesh Pyrolysis Apparatus. Review Of Scientific Instruments, 1989. 60(6): p. 1129-1139.

57. Quah, E. B. H. and C. Z. Li, Roles of desorbed radicals and reaction products during the oxidation of methane using a nickel mesh catalyst. Applied Catalysis A-General, 2004. 258(1): p. 63-71.

58. Quah, E. B. H., J. F. Mathews, and C. Z. Li, Interinfluence between reactions on the catalyst surface and reactions in the gas phase during the catalytic oxidation of methane with air. Journal Of Catalysis, 2001. 197(2): p. 315-323.

59. Westerhout, R. W. J., et al., Examination and evaluation of the use of screen heaters for the measurement of the high temperature pyrolysis kinetics of polyethene and polypropene. Industrial & Engineering Chemistry Research, 1997. 36(8): p. 3360-3368.

60. Wampler, T. P., Practical applications of analytical pyrolysis. Journal of Analytical and Applied Pyrolysis, 2004. 71(1): p. 1-12.

61. Wampler, T. P., Introduction to Pyrolysis-Capillary Gas Chromatography. Journal of Chromatography A, 1999. 842(1-2): p. 207-220.

62. Wampler, T. P., Applied Pyrolysis Handbook. 1995, New York: M. Dekker. x, 361 p.

63. Timofeeva, M. N., Acid Catalysis by Heteropoly Acids. Applied Catalysis A-General, 2003. 256(1-2): p. 19-35.

64. Arthur, C. L. and J. Pawliszyn, Solid-Phase Microextraction With Thermal-Desorption Using Fused-Silica Optical Fibers. Analytical Chemistry, 1990. 62(19): p. 2145-2148.

### SUMMARY OF INVENTION

One aspect of the invention is a method for identifying biological material containing non-volatile biomarker precursors. In summary, the method comprises (a) contacting the biological material with a catalyst, (b) heating to a temperature characteristic of catalysis to form volatile biomarkers, and (c) detecting and identifying the biomarkers. The temperature characteristic of catalysis will henceforth be referred to as a “catalytic temperature”, that is, one that at which catalysis occurs. Catalysis can occur at moderately low temperatures, i.e., temperatures substantially lower than temperatures required for pyrolysis. The biological material may be a material that comprises hazardous biomaterials, such as bacteria, bacterial spores, viruses, etc. The method is designed to derive volatile biomarkers from the biological material to determine the identity of the biological material at relatively mild, selective conditions.

Unlike pyrolytic methods, the method of the present invention does not require high pyrolysis temperatures to create the volatile biomarkers. Pyrolysis temperatures are temperatures that would be required using pyrolytic methods to create biomarkers. Typical pyrolysis temperatures exceed 350-400 degrees centrigrade (° C.) and range up to 750-800° C. The temperatures required for the present method are catalytic temperatures, where the predominant production of biomarkers is through catalysis. Typically, catalysis temperatures are temperatures that without a catalyst are incapable of creating readily-detectable biomarkers through pyrolysis from a biological material (readily-detectable refers to sufficient concentrations and heating rates to enable rapid, definitive detection). However, in the present invention the catalyst allows creation of biomarkers in sufficient concentrations and heating rates to enable fast, definitive detection at a catalytic temperature, which may be much lower than that required for pyrolysis, for example a temperature less than 200-300° C.

Advantages that accrue from the lower temperatures include lower power requirements, and the possibility of using smaller, low-power heating systems that are adaptable to hand-held systems. It is expected that lower temperatures will lead to fewer reactions and hence fewer side reactions.

In addition, since specific catalytic systems generally facilitate reaction to desirable products, it is expected that well-chosen catalysts will favor reactions that create desired biomarkers and will further create readily detectable concentrations of new biomarkers that are not produced in pyrolytic processes. For example, if derivatizing catalysts are used, volatile biomarkers in the form of derivative esters or other substances can be created rapidly at high concentrations under moderate conditions.

In certain applications, the higher selectivity and the higher speed of reaction using certain catalysts systems may be more important than operating at a low temperature, and practice of the invention in these instances may involve operation at or near temperatures used in prior-art pyrolytic systems. However, in the practice of the present invention the predominant generation of biomarkers is still through catalysis.

Biological materials refer to materials of biological origin that may or may not contain biologically hazardous components, such as biological spores or viruses, that the user wishes to detect and for which there are identified or potentially identifiable biomarkers. Of particular interest are the spores of Bacillus anthracis, Bacillus thuringiensis, and Bacillus subtilis var Niger. Also contemplated are materials that contain fatty acids, proteins, carbohydrates, deoxyribonucleic acid (DNA), lipids, peptidoglycans, and dipicolinic acid, that can create distinctive biomarkers that permit identification.

Contacting may occur in liquid phase or gas phase. The biological material to be tested, if it is a solid, may be dissolved in a liquid or suspended in a liquid or gas and contacted with a catalyst. The catalyst, as more fully described below, may be in the same phase as the dissolved or suspended sample, or may be a separate catalytically active surface in contact with a fluid in which the sample is dissolved or suspended.

As an example, volatile biomarkers may include one or more compounds, e.g., picolinic acid ester, and fatty acid methyl esters, created respectively from dipicolinic acid, and fatty acids, such as those found in lipids on spores and cells. In this instance, the catalyst is a derivatizing catalyst, such as an acid/base catalyst, that esterifies (e.g. methylates or ethylates) or otherwise derivatives the biological material into volatile biomarkers. Catalysts of this kind include superacid catalysts and other acidic or basic solids. An example of a suitable catalyst of the acid type is tungstophosphoric acid (H₃WP₁₂O₄₀). Solid acid catalysts, such as suitable zeolites are also contemplated.

Catalytic materials of the above-mentioned types can be made into any of a number of forms which permit sufficient contact of the biological material with the catalyst. Such catalyst-forms are well known in the art.

The catalyst may also be a decomposition catalyst. Such catalysts are typically porous, well-dispersed metals, metal oxides, and/or sulfides containing catalytically active surfaces that decompose organic compounds by breaking carbon-carbon, carbon-hydrogen, or carbon-oxygen bonds or other conceivable bonds in the molecule. Biomarkers or biomarker precursors expected to be generated by such catalysts include those detectable by pyrolytic methods or capable of further catalytic processing with another catalyst type to detectable biomarkers.

A decomposition catalyst may be finely-divided metal particles, metal particles dispersed in a porous support or carrier and/or a metal coated upon a solid surface or support. The construction should be such to ensure contact with the biological material, and the non-volatile biomarker precursors. Any suitable support, e.g. ceramic, carbon, or molecular sieve, is contemplated, including but not limited to porous ceramics in the form of particles, pellets, coated or solid monoliths, other shapes with channels, and other structured catalytic materials. The metal component of the decomposition catalyst may include one or more of noble or base metals, such as Co, Fe, Pt, Ni, Pd, or Rh.

The system for heating the sample may be a variation on any suitable heating technology and may include heated plates, ceramic membranes, rods, wires, or the like, and may comprise heating elements of electrically resistive metal, ceramics, and the like. It may be incorporated with the catalytic system, such as the wire-mesh system described below, configured to surround the catalyst, and/or place upstream or downstream of the catalyst to heat a flowing gas or vapor containing the sample. Heaters similar to those used in pyrolytic systems may be used, but will generally be smaller because the temperature and power consumption requirements are much lower for the method of the present invention.

Once produced, biomarkers may be detected/analyzed any suitable detection system including analytical chemistry techniques such as gas chromatography and mass spectrometry.

Another aspect of the invention is an apparatus that facilitates production of biomarkers from a biological material and comprises a reaction zone for contacting the biological material with the catalyst and derivatizing agents. The reaction zone will typically contain a catalyst and heating system to heat the biological material to a catalytic temperature. A collection zone is provided for collecting biomarkers for subsequent analysis or alternatively a detection zone is provided to detect and identify the biomarkers produced. The apparatus can be constructed for heating and catalytic reaction in gas or liquid, and may include either or both a derivation and decomposition catalyst.

In an aspect of the apparatus of the invention, the heating system may comprise a metal mesh that is electrically heated by passing a current through the mesh. The mesh may also provide a catalytically active surface. The mesh may be flat, curved or coiled, be single layered, multilayered, or constructed as a metal foam. The mesh can be constructed to provide a good means of distributing liquid samples across the heated (catalyst) surface, provide a high surface area for contact with the catalytic and/or heating surface. It can also provide for a means of drying solvents that may be required in the sample being tested. The catalytic function may be provided by its intrinsic material of construction, (e.g. Ni) or by coatings of an catalytically active metal (e.g. Pt, etc.) The mesh provides for flexible construction to provide the optimum or best mesh orientation, wire size, at the like.

### Catalysis

A catalyst is a material that lowers the activation barrier required for the formation of the desired products in a given chemical reaction, allowing it to proceed at rates many orders of magnitude more rapidly than is otherwise possible and with much higher selectivity. The catalyst is not consumed in the process but rather cyclically restored to its initial state during reaction (this process is called a turnover); commercial catalysts are capable of several million turnovers before requiring replacement. Heterogeneous catalysts typically consist of small crystallites of metal, metal oxide, or metal sulfide (the active phase) dispersed on a porous ceramic material, called a support. Oxide catalysts may include acidic solids such as zeolites, and super acids capable of catalyzing many different kinds of rearrangements of organic and biological compounds. Catalysts have found numerous applications in petroleum refining, chemicals manufacturing, and pollution control. Their major benefits are three-fold [47]. First, they facilitate reaction at low temperatures and pressures, thus dramatically lowering energy requirements for chemical reactions and processes. Second, they offer tremendous increases in selectivity and speed for a desired reaction or set of reactions. Third, they reduce the required equipment (especially reactor) volume.

### Application of Catalysis to the Generation of Biomarkers

In is believed, based upon a search of the scientific literature and patents, that there are currently no applications of heterogeneous catalysts for the production of biomarkers from bacterial spores. Catalysts can break the same types of bonds that are broken during pyrolysis, but at milder conditions. These include the breaking of carbon-carbon, carbon-nitrogen, and carbon-oxygen bonds. Catalysts used to break hydrocarbon carbon-carbon bonds include solid acids in the catalytic cracking of heavy hydrocarbons, metal (Ni, Pt, Rh) catalysts used in the steam reforming of hydrocarbons, and a combination solid acid zeolite/metal (Ni, Pt) catalyst for the hydrocracking of polynuclear aromatic hydrocarbons [47]. Metals that catalyze the breaking of carbon-sulfur, carbon-oxygen and carbon-nitrogen bonds are not readily found in the literature; however, metal sulfides are effective catalysts for these types of reactions.

Acid/base catalysts are known to catalyze derivatation, esterification and methylation reactions similar to the methylation reactions observed in spore pyrolysis. For example, superacid catalysts, in a homogenous (liquid-liquid) application, have been used in transesterification reactions for the breaking and reforming of carbon-oxygen bonds [48-52]. A study of a catalyst for transesterification of DPA, a common reagent in the pharmaceutical industry is reported [48].

The application of catalytic process to the generation of biomarkers from biological material, including bacterial spores, lowers the heat (energy) required and increases both the speed of formation and selectivity for the biomarkers. A comprehensive literature search has provided data to show that (1) the application of catalysis to the breakdown of bacterial spores has not been previously explored, (2) nickel and platinum catalysts have potential for breaking carbon-carbon bonds, and (3) heteropolyacid (superacid) catalysts such as tungstophosphoric acid (H₃WP₁₂O₄₀) have potential for the transesterification (methylation) of fatty acids.

An aspect pf the present invention is the application of catalysts for the generation of biomarkers from biological material, including bacterial spores at significantly milder conditions and with greater selectivity than previously used methods.

### BRIEF DESCRIPTION OF DRAWINGS

FIG. 1 is a schematic showing pyrolytic degradation and electron impact fragmentation pathway of dipicolinic acid [19].

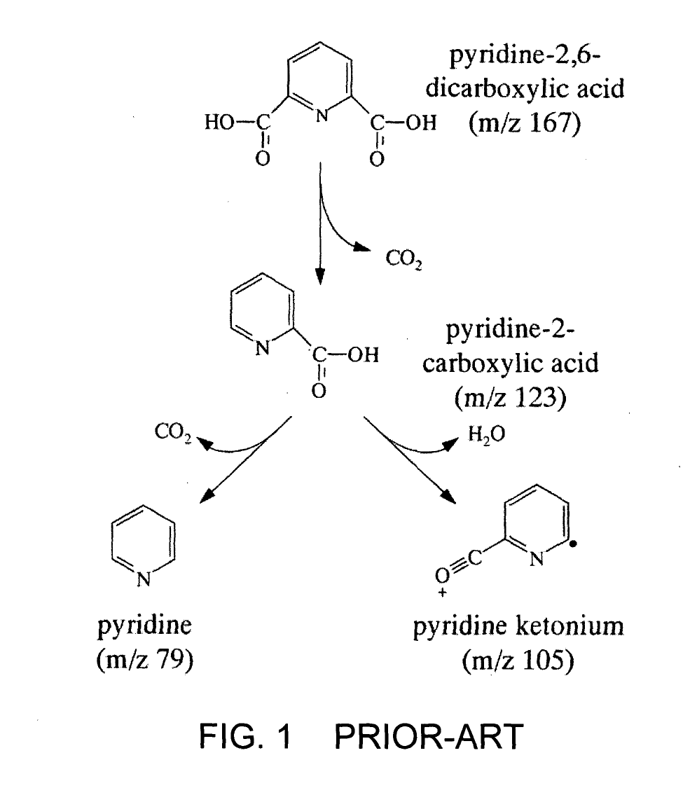

FIG. 2 is a graph showing the effect of temperature in the conversion of palmitic acid to its methyl ester.

FIG. 3 is a graph showing the reaction time for conversion of palmitic acid to its methyl ester.

FIG. 4 is a graph showing effect of molar ratio of methanol for conversion of palmitic acid to its methyl ester.

FIG. 5 is an MS spectra showing presence of picolinic acid methyl ester.

FIG. 6 is an MS spectra showing results from the decomposition of an anthrax spore.

FIG. 7 shows MS spectras showing results from the decomposition of sample bacterial spores.

FIG. 8 shows MS spectras showing results from the decomposition of sample bacterial spores.

FIG. 9 shows MS spectras showing results from the decomposition of sample bacterial spores.

FIG. 10 shows MS spectras showing results from the decomposition of sample bacterial spores.

FIG. 11 is a schematic diagram of an embodiment of an apparatus of the invention.

FIG. 12 is a schematic diagram of an alternate embodiment of an apparatus of the invention.

FIG. 13 is a schematic diagram of an alternate embodiment of an apparatus of the invention.

FIG. 14a and FIG. 14b are (a) isometric view and (b) front plane cut away view of a components of an apparatus of the invention using a wire mesh.

FIG. 15 is a view of an embodiment of an apparatus of the invention.

FIG. 16 is a detail of a portion of the apparatus of FIG. 15 showing the wire mesh screen mounted in the top flange of the apparatus.

### DETAILED DESCRIPTION

### Experimental Results

To demonstrate catalytic production of biomarkers from biological materials, experiments were conducted, which included the esterification of fatty acid and dipicolinic acid using a superacid (Tungsten phosphoric acid or TPA) catalyst, and decomposition of spores (BA, BG, BT—see Table 2) using TMAH and TPA.

TABLE 2
Bacillus Spores used in Catalyst Tests

| Species/Strain | Acronym |
|---|---|
| Bacillus anthracis | BA |
| Bacillus anthracis - Sterne strain | BASS |
| Bacillus thuringiensis | BT |
| Bacillus subtilis var Niger | BG |

Esterification of Fatty Acids and Dipicolinic Acid

Fatty acids are typical compounds produced during the decomposition of biological materials; fatty acids and dipicolinic acid are common compounds produced during the decomposition of bacterial spores. These acids are not easily detected due to their low volatility. However, the corresponding methyl esters can be detected easily after esterification using tungstophosphoric acid (TPA) as a catalyst. As mentioned above, biological material may be identified by the profile of FAMEs.

TPA is a promising candidate for the esterification of fatty acids; it is one of the Keggin-Structure heteropolyacids (HPAs or super acids), with a chemical composition of H₃PW₁₂O₄₀. In laboratory experiments, it demonstrated a highly catalytic activity for the esterification of fatty acids ranging from Lauric acid (C12 acid) to Stearic acid (C18 acid). This catalyst also demonstrated activity for the esterification of dipicolinic acid (DPA), which is another important biomarker for the detection of Bacillus spores.

A near-monolayer catalyst (as approximated from the molecular structure of TPA) was prepared by impregnating 50 wt % TPA on commercial silica support (308 m²/g). The prepared catalyst had a surface area 110 m²/g. The catalytic activity was not found to be affected by washing the catalyst with methanol, indicating a monolayer coverage of TPA (TPA is very soluble in methanol) in the catalyst. This also implies that the monolayer active component is stable on the surface.

Methylation of pure fatty acid compounds was catalyzed by silica-supported tungstophosphoric acid (TPA). The procedure utilized to demonstrate this varies slightly according to the solvent used (e.g. temperatures and drying times were varied). The general scheme was as follows: In a small vial, the model compound (usually palmitic acid) was dissolved in a solvent (usually water), to which methanol and a silica-supported TPA catalyst were added. 1-2 μL of the mixture was transferred to and heated in the pyrolyzer cup at low temperatures (e.g. below the boiling point of the mixture, ˜60° C. in the case of methanol) to drive off the solvent, following which the residue was heated rapidly to temperatures between 250-300° C. (temperatures in excess of 400° C. were found to degrade the reactants). The GC/MS data were examined for fatty acid and fatty acid methyl ester peaks. Hexadecane was added to the mixture to serve as an internal standard for quantitative analysis. It was found that the extent of palmitic acid methylation in the presence of methanol is significantly enhanced by TPA even at room temperature. These results were obtained with both water and octane as solvents.

Results—Fatty acids

Experiments with catalytic methylation of fatty acids (C₁₂-C₁₈) were conducted; methylation activity and selectivity of TPA with methanol was found to be similar for all fatty acids in this range. The results of palmitic acid, as an example, are discussed below.

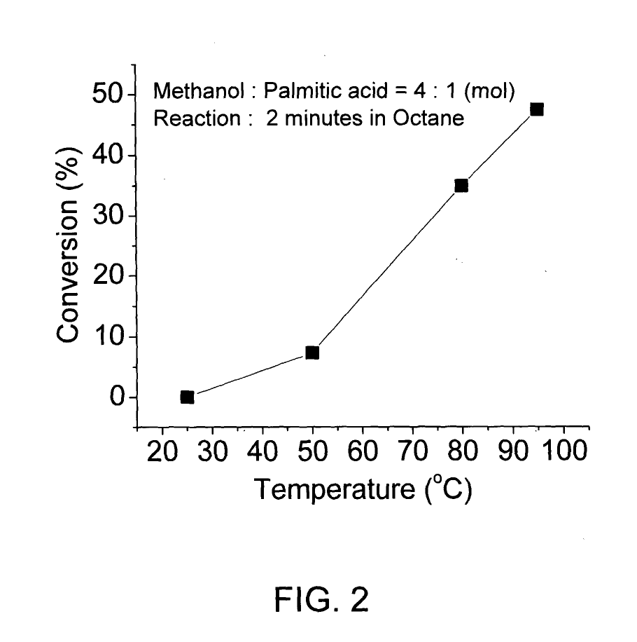

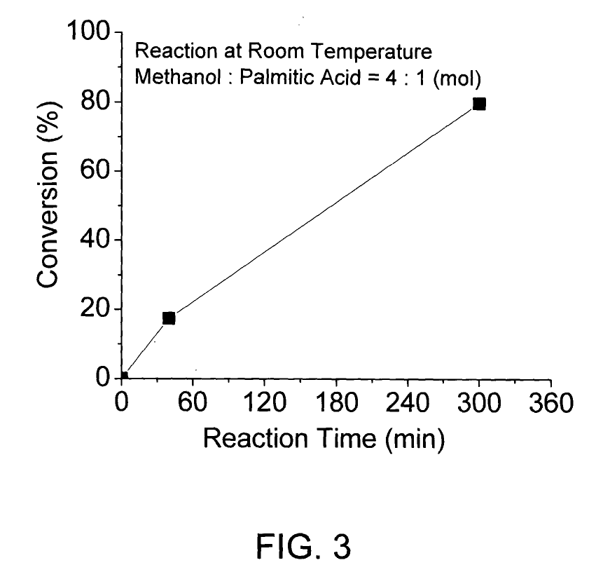

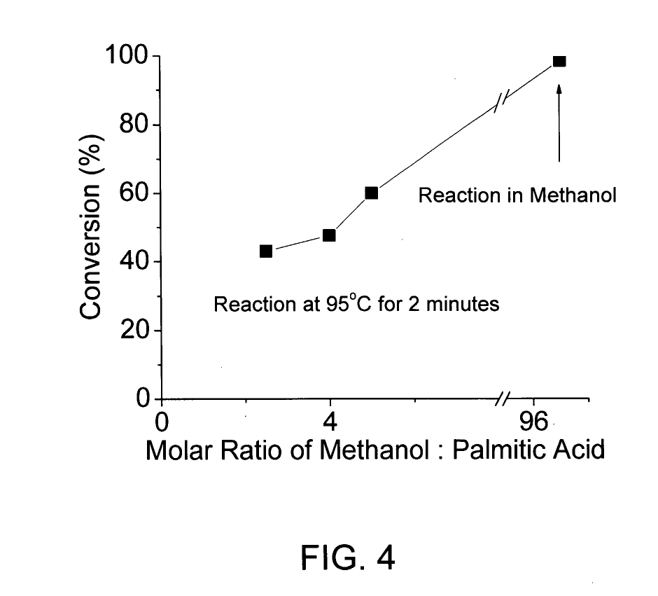

Referring to FIG. 2, the catalytic conversion of palmitic acid to its methyl ester is 50% after reaction for 2 minutes at 95° C. Referring to FIG. 3, the conversion increases to 80% when the mixture is at room temperature for more than 5 hours; however, the reaction does not occur appreciably at the same conditions without a catalyst. These results indicate that the catalyst is very active for the methylation of all types of fatty acids. Referring to FIG. 4, it is shown that reaction conversion is greater at a high ratio of methanol/palmitic acid. The reaction almost is complete at 95° C. for 2 minutes when using methanol as the solvent in a sealed tube to prevent solvent loss.

Results—Dipicolinic Acid

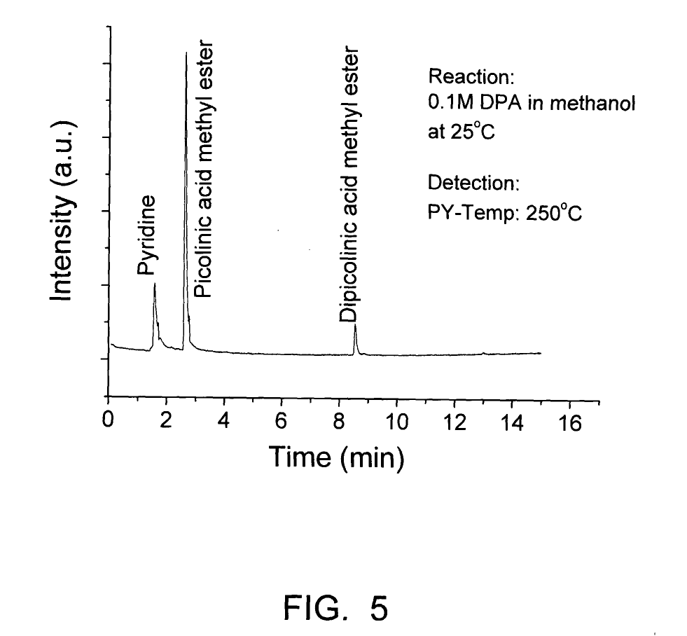

Referring to FIG. 5, it is shown that dipicolinic acid is converted to its methyl ester or picolinic acid methyl ester at 25° C. under the same catalytic conditions.

Decomposition of BA, BASS, BG and BT Spores

Samples of the stock spore suspension were vortexed and an aliquot was transferred, along with other reagents and/or catalysts (e.g. TMAH, TPA, methanol, etc.), into a small eppendorf tube. Approximately 2 μL were used. An appropriate GC method for fatty acid (and fatty acid methyl ester) profiling was utilized for analysis of the produced FAME profile.

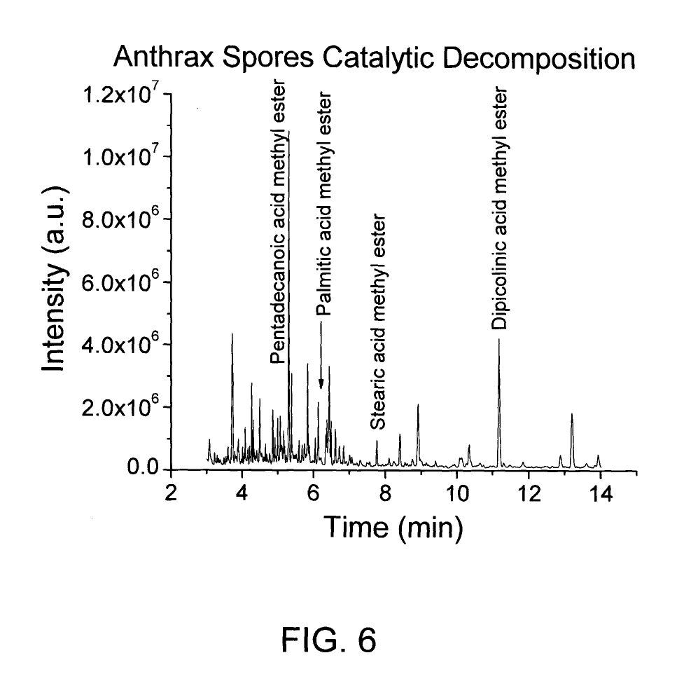

Referring to FIG. 6, the results from the decomposition of BA in the presence of TMAH and TPA indicate that the significant amounts of biomarkers were produced. It is also seen that the reaction was selective, i.e., not creating numerous by-products that create noise and interfere with the detection and identification of biomarkers.

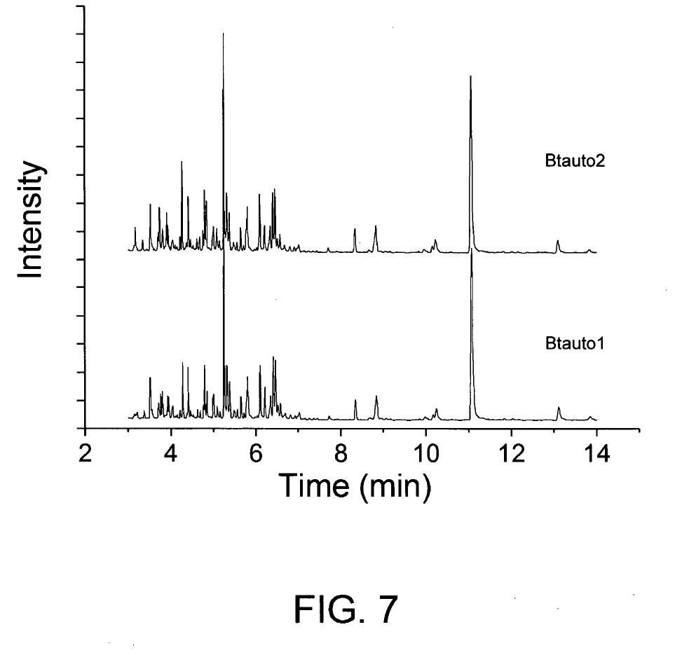

Referring to FIG. 7, the results of spore (autoclaved BT) decomposition in TMAH and TPA were similar for separate experiments, indicating that the results are reproducible.

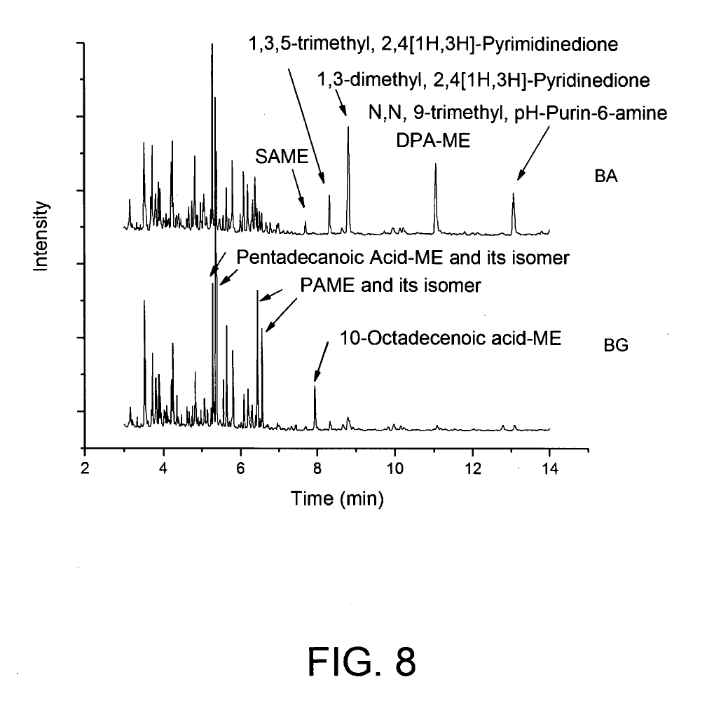

Referring to FIG. 8, the results of the decomposition of BG and BA spores in the presence of TMAH and TPA indicate that the species can be distinguished by the unique pattern of catalytically-produced biomarkers (fatty acid methyl esters or pyridine derivatives) for each spore.

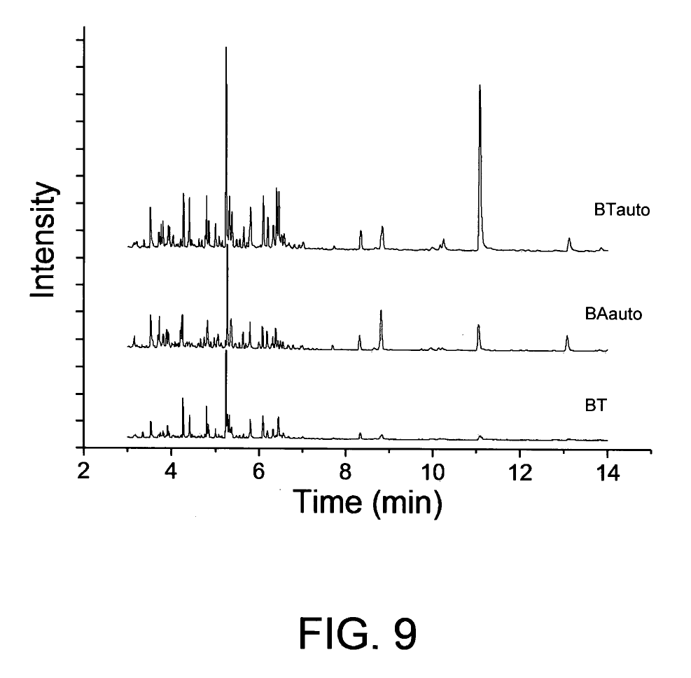

Referring to FIG. 9, similar results (in terms of peak locations) are found in the spectra of BT and BA with TMAH and TPA. However, the intensities of the peaks change significantly, indicating that that autoclaving has a strong impact on the results.

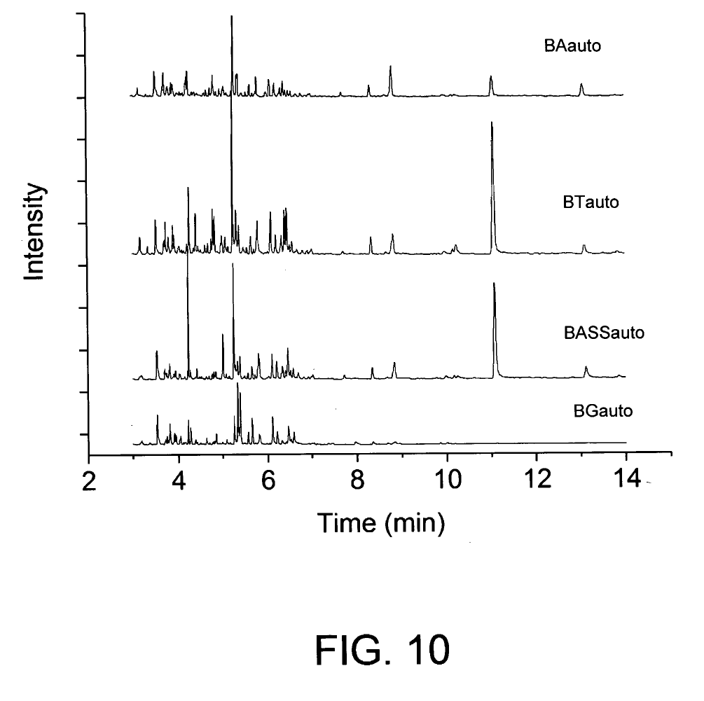

Referring to FIG. 10, the spectra from the decomposition of autoclaved spores (BA, BT, BASS, BG) are all visibly different, showing that differentiation between species is possible by this method.

Summary of Results and Conclusions

The catalytic esterification of fatty acids and dipicolinic acid by a superacid catalyst is demonstrated. As fatty acids and dipicolinic acid are typical compounds generated in the decomposition of bacteria and bacterial spores, this supports the claim that catalysis can generate biomarkers from biological material.

The decomposition of BA, BT, BG and BASS spores under mild conditions in the presence of a methylating agent (TMAH) and catalytic material (TPA) was shown to produce biomarkers capable of distinguishing between and identifying Bacillus species.

As metals such as Pt, Ni, and Pd have been shown in previous studies to catalyze breaking of C—C, C—N and C—O bonds, it follows that they can catalytically produce hydrocarbon fragments via spore decomposition that are likely to be biomarkers or biomarker precursors.

### Apparatus

A handheld biomarker generator (HBG) is a portable device, necessitating special design requirements. This includes, for example methods of sample collection, use of decomposition and derivatization catalysts and reagents, device geometry and construction, etc.

The principal purpose of a HBG is to release a sufficient quantity of important chemical biomarkers from a sample, which may be, for example, Bacillus anthracis endospores, but can be any biological or other small biochemical compounds (e.g. proteins, DNA, sugars, etc.) and deposit an adequate quantity of the released biomarkers for a detection and identification system, such as a deposit on an solid phase microextraction (SPME) fiber to be detected by GC/MS. An alternative to the SPME fiber is the use of a direct connection to the GC/MS A suitable HBG should
Be portable, (probable scenario: a small container, which includes a battery, electronics control, etc., can accept removable, disposable cartridges that can be bagged and saved after use)
Be handheld,
Be re-usable, consume low power (i.e. <75 W)
Assist in the collection of a sample (e.g. with cloth-like wipes, swabs, etc.)
Include any necessary chemical reagents and/or catalysts
Assist in the proper dispersion, mixing, heating, etc. of a sample and necessary reagents
Incorporate a sample collection device such as a SPME (solid-phase microextraction) fiber (either packaged inside the unused device or inserted during/after use) for transferring the produced/volatilized biomarkers to the detector.

### Example A—Apparatus

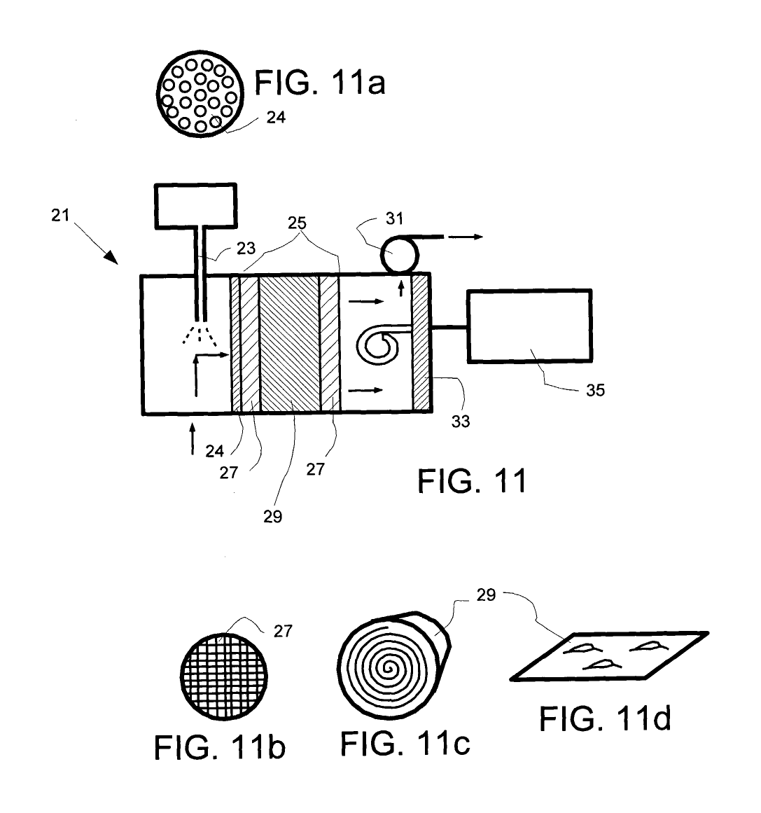

A schematic diagram of an HBG 21 embodying the invention is presented in FIG. 11. An injector syringe or spray nozzle 23 disperses the liquid sample, which is carried by an air stream (shown by flow arrows) through a perforated plate 24 (FIG. 11a) into a “catalytic zone” 25 comprised two catalysts: the first is a wire mesh 27 (referred to as Catalyst 1 or the decomposition catalyst) consisting of bare or Pt-coated nickel and serves to thermally and catalytically break down the spores, releasing volatile spore components. (See also FIG. 11b) The second catalyst 29 (referred to as Catalyst 2 or the derivatization catalyst) esterifies organic acid compounds produced during spore decomposition and be coated on the surface of a dimpled metal foil monolith. (See also FIG. 111c & 11d) Resistively heating the mesh produces the thermal energy necessary to break apart the spores and to heat the metal monolith (the latter being highly heat-conductive and thus able to be heated by the wire mesh) to provide energy for the esterification reactions. Air (filtered to 0.1 μm upon entering the device to remove potentially interfering particulates) is evacuated from the back or exit side of the HBG by a mini-diaphragm pump 31 to draw spores and decomposition products through the catalysts and expel the air. Organic compounds produced catalytically are absorbed through an “organics-permeable membrane,” which is the interface 33 to a collection/detection section 35, e.g. a GC/MS (gas chromatograph/mass spectrometer, or a VGC/ITMS-vacuum gas chromatograph/ion trap mass spectrometer).

### Example B—Apparatus

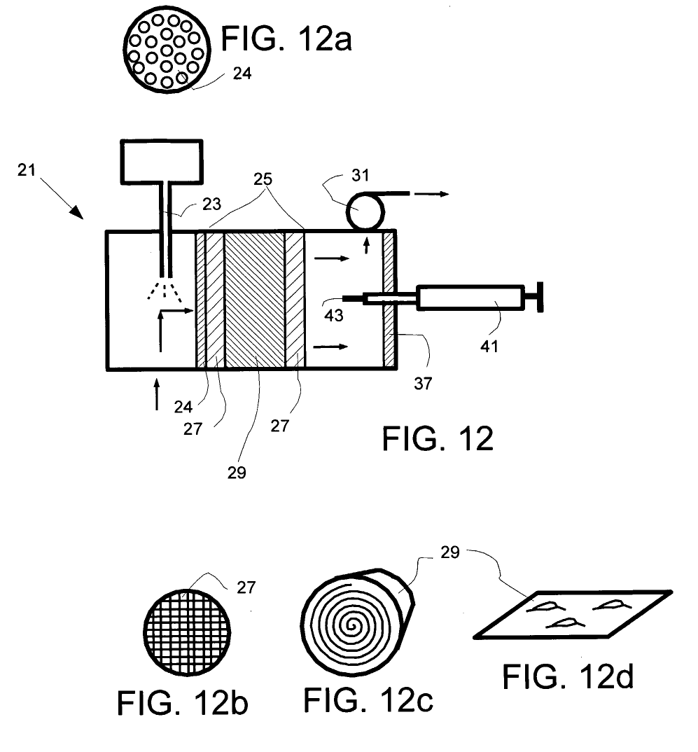

Reference is now made to FIGS. 12, 12a, 12b, 12c, and 12d. This is a variation on the design of FIG. 11, using the same reference numbers where applicable. The most important change is the use a SPME fiber 43 to absorb and transfer the biomarker compounds to the GC/MS. In the illustrated design the SPME retracts into a syringe-like needle 41 that extends through a septum 37, but the device is not a syringe in the traditional sense of the word.

### Example C—Apparatus

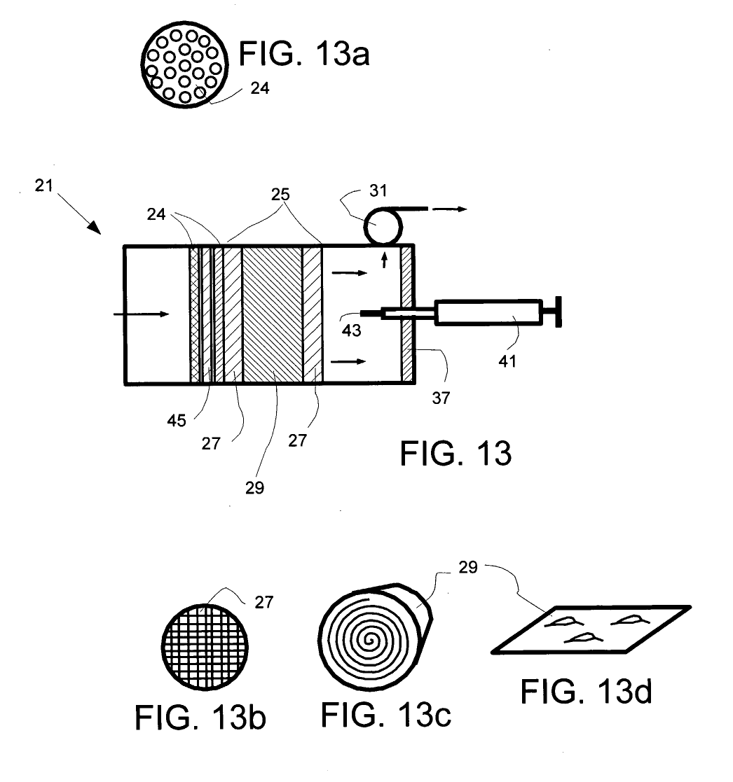

Reference is now made to FIGS. 13a, 13b, 13c, 13d, which is a variation of FIG. 12 and FIG. 11, using the same reference where applicable. The main difference is the use of a ceramic wipe 45 between two porous plates or mesh 24 to collect solid samples. This wipe can incorporate either or both of the decomposition or derivatization catalysts, as well as necessary reagents. Following sample collection, the wipe is sandwiched between two heated meshes to facilitate catalysis and production of biomarkers.

### Example D—Apparatus

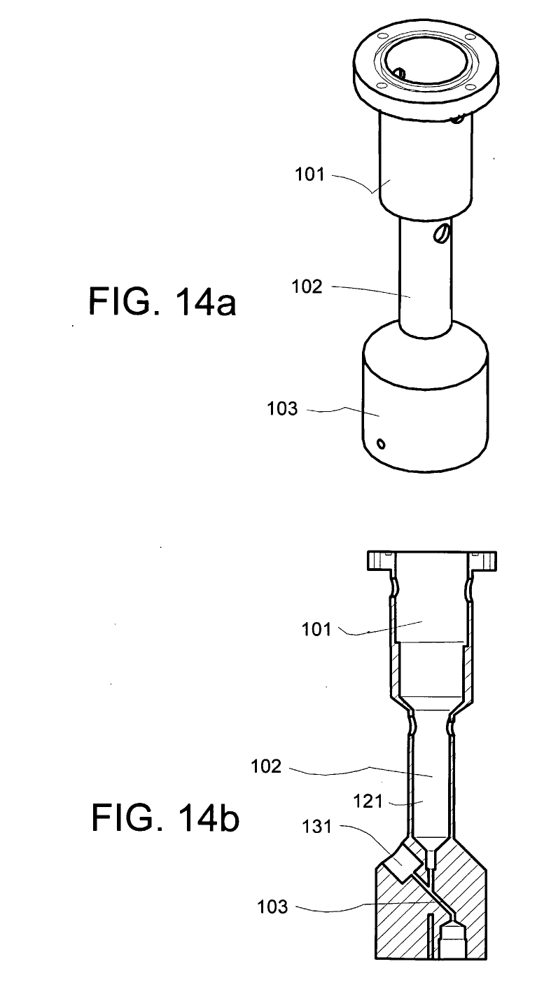

A prototype of the HBG (often referred to the “wire mesh test unit,” or WMTU) was constructed to evaluate the processes of the invention. FIG. 14a and FIG. 14b shows the major part of this device.

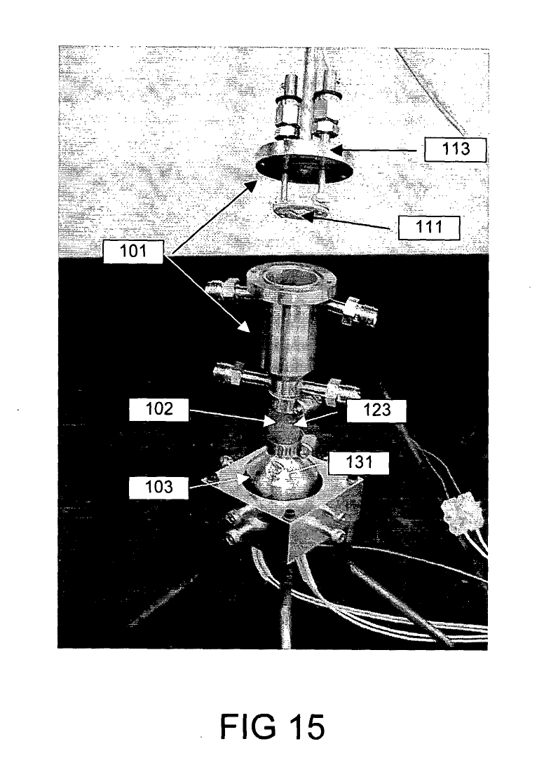

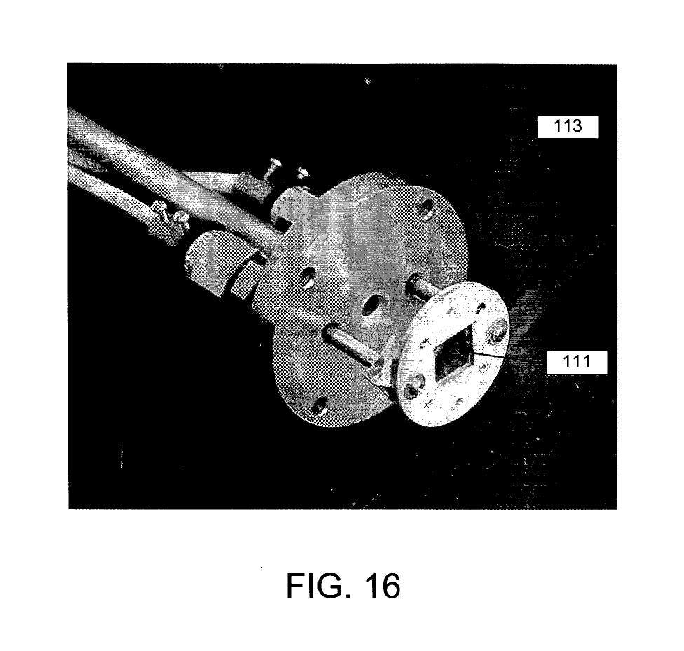

FIG. 15 and FIG. 16 are prespective views of the apparatus. The device consists of three zones as indicated in FIGS. 14a, 14b, and 15:
Zone 1: (101) Location of derivatization catalyst, or the wire mesh/heater
Zone 2: (102) Location of decomposition catalyst, the methylation catalyst
Zone 3: (103) Location of chemical product transfer to the SPME fiber (which is inserted into the port 131 (FIG. 14b).

Zone 1:

A very fine nickel wire mesh 111 (FIGS. 14 and 16) is used to collect and resistively heat spores, catalyst, and/or reagents that may be used to abet spore decomposition and biomarker production. The setup of the wire mesh holding device 113, which is inserted into the top of the WMTU, is shown in FIG. 16. Any suitable system is contemplated for making the electrical connections for heating, for holding the mesh in place, introduction of the sample into the device, and directing the flow through the device.

Zone 2:

Zone 2 is designed to incorporate cylindrical monoliths or powdered catalysts. Shown is the chamber 121 (FIG. 14a) for containing monoliths or catalysts. To heat the chamber of Zone 2, a glass-insulated nichrome wire 123 (FIG. 15) is wrapped around the outside, although an aluminum heating block may used. Any suitable system is contemplated for electrical connections to heat by any suitable method and for controlling the temperature (e.g. a temperature control system utilizing temperature controllers with the appropriate tuning parameters).

Zone 3:

This section, which is the location of chemical product transfer to the SPME fiber, (See port 131) will be heated if the WMTU is mounted on top of a GC injection port.

Ultimately, a miniaturized version of the HBG is contemplated.

### Example E—Liquid-Based Reactions

In the above example of the apparatus and method of the invention, the biological sample, reagents, and products are dry, i.e. surrounded by air or other gas. Alternately, the reactions can be liquid-based with dissolved compounds/reagents and suspended Bacillus endospores of various species. The following is described a summary of liquid-based procedures.

First, methylation of pure fatty acid compounds (representative of spore decomposition products) is catalyzed by silica-supported tungstophosphoric acid (TPA). Although the procedure utilized to demonstrate this varies slightly according to the solvent used (e.g. temperatures and drying times vary), the general scheme is as follows: In a small vial, the model compound (usually palmitic acid) is dissolved in a solvent (usually water), to which methanol and a silica-supported TPA catalyst are added. 1-2 μL of the mixture is transferred to and heated in a pyrolyzer cup at low temperatures (e.g. below the boiling point of the mixture, ˜60° C. in the case of methanol) to drive off the solvent, following which the residue is pyrolyzed at temperatures between 250-300° C. (temperatures in excess of 400° C. degrade the reactants, including the TPA). The GC/MS data reveal that palmitic acid methylation in the presence of methanol is significantly enhanced by TPA even at room temperature. These results are the same with both water and octane as solvents.

Second, tests are made to identify the products that are produced under various liquid-based treatments of spores. The stock spore suspension is vortexed and an aliquot is transferred, along with other reagents and/or catalysts (e.g. TMAH, TPA, methanol, etc.), into a small eppendorf tube for mixing. About 10 μL of the resultant mixture is transferred into a small glass capillary tube that has been heat-sealed on one end. After this transfer, the other end of the glass capillary is sealed, and the spores are heated to temperatures up to at least 200° C. Following this heat treatment, the capillary is broken and approximately 2 μL is removed and added to the pyrolyzer cup. An appropriate GC method for fatty acid (and fatty acid methyl ester) profiling is utilized for analysis of the resultant chemical profile.

### Variants in the Apparatus Design

Many suitable variants of the HBG design are suitable for practice of the invention. An example of a suitable HBG would have the following features, each of which is described in more detail below.

1. Sample collection from a source (contaminated surface, powder, and/or liquid)

2. Sample presentation/introduction to the HBG

3. Initial sample decomposition via heating and catalysis

4. Conversion of the chemical products from 3 above to more volatile, stable species (e.g. esterification of fatty and other organic acids)

5. Collection of the products from 4 above onto a SPME fiber

6. Retraction and withdrawal of the SPME fiber for analysis by GC/MS

Sample Collection

There are two main expected forms of sample unknowns: powder (e.g. weaponized anthrax spores) and liquid suspensions. Collecting a powdered sample may be accomplished by using a swab or wipe. The wipe may be attached to or inserted into the device, which could consist of two parts that screw or snap together around the wipe after sample collection. Liquid samples may be collected in a syringe and injected or sprayed into the device, or the device itself may be used to absorb or otherwise take up liquids. The swab or wipe may also be used with liquid samples in a similar fashion (i.e. spray the liquid onto the wipe or use the wipe to soak up the liquid).

Sample Presentation

A wipe can be sandwiched between the wire mesh heaters in the device and heated directly, or it may be inserted into a liquid reservoir containing the reagents and catalysts. With sufficient air flow and/or heating, the spores and spore products may dislodge from the wipe and pass onto a heated (optionally catalytic) mesh and/or through the system.

Alternatively, liquid samples may be injected, sprayed, or squirted into the device, which will have appropriate channels and geometry to direct the liquid so that it is rapidly mixed, heated, and dried (if necessary) and so that particulates/spores present in the liquid deposit only on the desired heated or catalytic surface (such as a wire mesh). Any high surface area material (mesh, foam, etc.) may be used to collect, disperse, and dry (as necessary) the liquid.

Initial Sample Decomposition

A combination of heating and catalysis may be used. The decomposition catalyst may take the form of any material that aids in the thermal or chemical degradation of the of biological material into biomarkers, e.g., degradation of the spore to release/volatilize fatty acids, dipicolinic acid, protein fragments, and any other unique chemical biomarker compound(s).

If the catalyst includes a metal such as Ni and Pt (functional in breaking C—C bonds), the catalyst may stand alone or be plated onto a wire mesh or other high surface area material (such as a nickel or other metal foam). Alternatively, nano-clusters of these materials may be dispersed on the outside of the spores, essentially bringing the catalyst to the spores (rather than the spores to the catalyst). The process for dispersing the catalyst over the spores can be built into the device. Nanoclusters or other catalysts/reagents may be included in the wipe described above in order to facilitate the coating of the spores with the catalyst, or may otherwise be incorporated into the mesh or final device. The initial sample decomposition may be done in a liquid mixture, or via decomposition of dried spores.

The heater portion of the device can be any suitable system. A suitable system has been found to be fine, electroformed nickel wire meshes that have 200 and 1500 openings per inch (available from Precision Eforming). Pieces of the mesh with 200 holes-per-inch have been resistively heated to red-hot. Normally these mesh materials are used for electrical shielding for application in sensitive electronics applications as well as sieve materials. It is believed that these mesh materials have not been used as a heater, where electroformed wire mesh is used as a mini-heater or mini-pyrolyzer for the production of biomarkers from biological material. Wire meshes have been used as pyrolyzer and catalytic devices, but not for the production of biomarkers. The heated wire mesh apparatus was invented in the 1950's by Loison and Chauvin [53] and has received subsequent use, particularly in the area of coal particle pyrolysis/volatilization. Research work has not only focused on the pyrolysis reactions of coal (for examples, see [54, 55]), but also on the effect of mesh heating rates on pyrolysis product yield [56], the catalytic effects of an electrically heated nickel mesh [57, 58] on methane oxidation in air, and polyethylene and polypropylene pyrolysis kinetics [59].

The mesh, since it is constructed of very fine wires (from 10-200 microns in diameter), may be heated more rapidly than current larger-scale pyrolyzers at lower levels of power consumption. This is an advantage to the field of pyrolysis; the literature has reported that rapid heating is desirable for pyrolyzer design [60-62].

Furthermore, depending on the wire dimensions and hole sizes, the fine mesh can present more total surface area than a flat solid alone (which is the current design of all commercial pyrolyzer devices known to the applicants; e.g. curie point, resistively-heated wires, heated metal foil, and heated crucible-type pyrolyzers), improving dispersal and heating of fine particulate matter (including bacterial vegetative cells and endospores) that may clump together upon drying. Lastly, the use of one or more layers of the fine mesh as a filter during sample introduction/collection has merit.

Any suitable power source for the heater is contemplated, which may include various ways to drive the current, for example, either parallel to one set of the wires and perpendicular to another, or diagonal through both sets of wires at the same time. Also, different mesh pattern geometries (e.g. hexagonal) or multi-layer designs are contemplated. Multi-layer designs and the use of a metal “sponge” rather than a mesh is contemplated. The surface of the mesh may also include special coatings (e.g. nano-crystallites) or dendrites that assist in spore degradation. The mesh may be laid flat, or the mesh rolled in a cylindrical shape and the electrical current applied along its axis. This might enhance product transport characteristics, would place more surface area of the mesh nearer to the SPME fiber (see below), and may assist in initial sample deposition (e.g. a swab might be passed through the center of it, depositing sample material along the inside).

Conversion of Decomposition Products

Biomarker products generated by the decomposition catalyst (which may include fatty acids, dipicolinic acid, and/or other biomarkers) may undergo subsequent reactions over the derivatization catalyst, such as tungstophosphoric acid [63] supported on silica, although other formulations may be used). The products of these reactions are expected to be esters, but may be other compounds. The device will contain necessary reagents (or a means to introduce them) for these reactions (e.g. methanol, TMAH, etc.). This catalyst and its reagents may be incorporated into the collection wipe, decomposition mesh, or a separate monolith or mesh. They may also be incorporated into a specialized SPME fiber (eliminating dead volume).

Collection of Products onto SPME Fiber

SPME is short for solid phase microextraction and is a technology that allows for the adsorption of sample onto a modified solid support during collection (either from dipping in a liquid solution or exposing to gas containing volatile compounds of interest), and subsequent desorption of the sample either with a solvent or by thermal means [64].

The SPME fiber (which retracts into a needle that can be inserted inside analytical equipment such as GC/MS) may be initially present in the collection-/reaction device and extended during reaction time, or may be extended or inserted after sample introduction and/or initial treatment. The SPME fiber may be protected from adsorption of larger chemical fragments (or those of a slower diffusivity than the desired compounds) by a protective outer sheath. Finally, perhaps the SPME fiber could be impregnated or otherwise loaded with a catalyst material.

Retraction and Withdrawal of SPME Fiber

The fiber will be retracted into its protective needle sheath and the syringe withdrawn in order to transfer the products to an analytical instrument.

Other Process Notes

To transport biomarkers from the catalyst to the SPME fiber, a miniature diaphragm pump may be used. Such devices are produced and commercially available. (e.g. www.virtualpumps.com). A pump is not used in instances where the process does not involve flow, where reaction takes place in a liquid suspension or in a small, simple reaction chamber

In addition, the surfaces of any parts of the device that contact spores, reagents, biomarkers, etc. may receive special treatments (e.g. chemical, heat, etc.) or coatings. Such treatments will be used, as necessary, to minimize/maximize the physical attraction, chemical adsorption, or charge attraction/repulsion of the spores and chemical species present to improve device efficiency.

While this invention has been described with reference to certain specific embodiments and examples, it will be recognized by those skilled in the art that many variations are possible without departing from the scope and spirit of this invention, and that the invention, as described by the claims, is intended to cover all changes and modifications of the invention which do not depart from the spirit of the invention.

What is claimed is:

1. A method for identifying biological material containing volatile and/or non-volatile biomarker precursors, the method comprising:
contacting the biological material with a catalyst;
heating to a catalytic temperature to form volatile biomarkers;
detecting and identifying the biomarkers.

2. A method as in claim 1 wherein the biological material contains bacterial spores.

3. A method as in claim 1 wherein the biological material contains one or more of spores, bacteria, virus, and toxin.

4. A method as in claim 1 wherein the biological material contains one or more spores selected from Bacillus anthracis, Bacillus thuringiensis, and Bacillus subtilis var Niger.

5. A method as in claim 1 wherein the biomarker precursors include one or more of, fatty acids, proteins, carbohydrates, deoxyribonucleic acid (DNA), lipids, and dipicolinic acid.

6. A method as in claim 1 wherein the contacting is in a liquid phase or a gas phase.

7. A method as in claim 1 wherein the volatile biomarkers include one or more of picolinic acid, and fatty acid methyl esters, and the catalyst is an acid/base catalyst.

8. A method as in claim 1 wherein the catalyst is a derivatization catalyst to esterify the biomarker precursors.

9. A method as in claim 1 wherein the catalyst is a superacid catalyst and the volatile biomarkers are formed by derivation of fatty acids.

10. A method as in claim 1 wherein the catalyst is a superacid catalyst and the volatile biomarkers are formed by methylating fatty acids.

11. A method as in claim 1 wherein the catalytic temperature is less than temperatures required for pyrolysis of the biological material.

12. A method as in claim 1 wherein the catalytic temperature is less than 300 degrees centigrade.

13. A method for identifying biological material containing non-volatile and volatile biomarker precursors, the method comprising:
contacting in liquid phase the biological material with a super acid catalyst;
heating to a catalytic temperature to methylate the non-volatile biomarker precursors to form methylated-ester biomarkers;
detecting and identifying the methylated-ester biomarkers.

14. A method as in claim 13 wherein the non-volatile biomarker precursors comprise fatty acids and the methylated volatile biomarkers comprise fatty acid methyl esters.

15. A method as in claim 13 wherein the non-volatile biomarker precursors comprise dipicolinic acid and the methylated volatile biomarkers comprise a methyl ester of dipicolinic acid.

16. A method as in claim 13 wherein the catalyst is tungstophosphoric acid (H₃WP₁₂O₄₀).

17. A method as in claim 13 wherein the biological material contains one or more spores selected from Bacillus anthracis, Bacillus thuringiensis, and Bacillus subtilis var Niger.

18. A method as in claim 1 wherein the catalyst is a decomposition catalyst to break down biomarker precursors.

19. A method as in claim 1 wherein the catalyst is a metal decomposition catalyst and volatile biomarkers are formed by breaking carbon-carbon bonds.

20. A method for identifying biological material containing non-volatile biomarker precursors, the method comprising:
contacting in gas phase the biological material with a solid metal decomposition catalyst;
heating to a catalytic temperature to degrade non-volatile biomarker precursors to form volatile degradation products;
detecting and identifying the volatile degradation products.

21. A method as in claim 20 wherein the non-volatile biomarker precurors comprises one or more of fatty acids, protein, peptidoglycan, and DNA.

22. A method as in claim 20 wherein the catalyst comprises one or more noble or base metals.

23. A method as in claim 20 wherein the catalyst comprises one or more of Pt, Ni, Pd, and Rh.

24. A method as in claim 1 wherein the detecting and identifying the biomarkers comprises analytical chemistry techniques selected from gas chromatography, mass spectrometry, and ion trap mass spectrometry.

25. A method as in claim 1 wherein contacting with the catalyst comprises contacting with decomposition catalyst to break down the biomarker precursors and contacting with a derivatization catalyst to esterify the biomarker precursors.

26. A method as in claim 1 wherein the heating comprises contacting with a heated metal mesh.

27. A method as in claim 1 wherein the heating and the contacting with a catalyst are both accomplished by contacting with a heated metal mesh having a catalytically active surface.

28. An apparatus for identifying biological material containing non-volatile and volatile biomarker precursors, the apparatus comprising:
a reaction zone with a catalyst constructed and configured for contacting the biological material with the catalyst and heating the biological material to a catalytic temperature to form volatile biomarkers;
collection for collecting the biomarkers for detection and identification.

29. An apparatus as in claim 28 wherein the reaction zone comprises first and second contacting and heating zones, the first zone comprising a decomposition catalyst to break down the biomarker precursors; the second zone comprising a derivatization catalyst to esterify the biomarker precursors.

30. An apparatus as in claim 28 wherein the collection zone comprises one or more of gas chromatography systems and mass spectrometry systems.

31. An apparatus as in claim 28 wherein the reaction zone comprises a metal mesh that functions as the heater.

32. An apparatus as in claim 31 wherein the metal mesh has a catalytically active surface and functions as the catalyst.

33. An apparatus as in claim 31 wherein the mesh is single-layered or multilayered or foam-like.

34. An apparatus as in claim 31 wherein the mesh in constructed to distribute liquid samples across the heated surface.
